(19) United States
(12) Patent Application Publication
(10) Pub. No.: US 2020/0357299 A1
(43) Pub. Date: Nov. 12, 2020

(54) ENHANCED REALITY REHABILITATION SYSTEM AND METHOD OF USING THE SAME

(71) Applicants: Amish Patel, Chicago, IL (US); Timothy Denison, Anacortes, WA (US)

(72) Inventors: Amish Patel, Chicago, IL (US); Timothy Denison, Anacortes, WA (US)

(21) Appl. No.: 16/652,217

(22) PCT Filed: Jan. 18, 2019
(86) PCT No.: PCT/US19/14199

§ 371 Date: Mar. 30, 2020

Related U.S. Application Data
(60) Provisional application No. 62/619,086, filed on Jan. 18, 2018.

(51) Int. Cl. G09B 19/00; G06F 3/01; A63F 13/80; A63F 13/212; A61B 5/16; A61B 5/00; A61B 5/11; A61B 5/0476; G16H 20/30; G16H 50/20

(52) U.S. Cl. CPC G09B 19/00; G06F 3/011; A63F 13/80; A63F 13/212; A61B 5/165; A61B 5/4836; A63F 2300/1012; A61B 5/11; A61B 5/0476; A61B 5/7264; G16H 20/30; G16H 50/20; A63F 2300/8082; A61B 5/4833

(57) ABSTRACT

A network-based rehabilitation system for treating ailments of a user utilizing an enhanced reality environment is provided. The system has an enhanced reality device wearable by the user, the device being in communication with a network and configured to enable a user to interact with the enhanced reality environment to execute a rehabilitation routine; at least one sensor configured to capture biometrics of the user, a condition of the user, or both, wherein the at least one sensor is in communication with the enhanced reality device and the network; a progress analysis module configured to analyze a rehabilitation routine performance of the user, and a routine modification module configured to adjust the rehabilitation routine based on the performance of the user, wherein the routine modification module executes a machine learning algorithm and recommends a routine adjustment based on the machine.

### CROSS-REFERENCE TO RELATED APPLICATIONS

The present utility patent application is a national stage application filed under 35 U.S.C. § 371 of International Patent Application No. PCT/US19/14199 filed on Jan. 18, 2019 entitled Enhanced Reality Rehabilitation System and Method of Using the Same which claims the priority benefit of U.S. provisional patent application Ser. No. 62/619,086 filed on Jan. 18, 2018, each of which the entire contents of are incorporated by reference herein for all purposes.

### FIELD OF THE INVENTION

The present invention relates generally to virtual reality-based routines and systems for use by individuals to treat chronic pain. More particularly, the present invention relates to certain new and useful advances in virtual reality systems that can be used by individuals alike to create an immersive and interactive rehabilitation program to help treat chronic pain, with adjustments being made to the virtual environment in real time via machine learning algorithms to optimize the treatment based on the patient's performance, reference being had to the drawings accompanying and forming a part of the same.

### BACKGROUND

According to a study conducted by the Institute of Medicine of the National Academies, 100 million Americans suffer from chronic pain. In the United States alone, chronic pain affects more Americans than diabetes, heart disease and cancer combined. The most common type of chronic pain is back pain, with lower back pain being the leading cause of disability worldwide and among the most common reasons for missed work.

It is estimated that 149 million days of work are missed each year due to chronic pain, which is defined as pain lasting longer than 3 months in duration and generally limits an individual's daily activities. Various studies have estimated that chronic lower back pain is prevalent amongst 15-45% of Americans and is the second leading reason for doctor's visits with direct costs of $50 billion a year and indirect costs as high as $100 billion per year.

The most common option for treating chronic pain today is through the use of opioids; unfortunately, there is an opioid epidemic and the harmful impact of treating chronic pain with opioids has resulted in addiction and in many cases death. This epidemic has affected families of all socioeconomic groups and these indirect costs are immeasurable.

Medical practitioners are often at the front line of treating patients with chronic pain and struggle to find tools that are effective. In particular, chronic lower back pain can be extremely difficult to treat owing to its pathophysiologic complexity. Practitioners for years have been left with no other options other than recommending invasive surgery or using opioids to quell the pain of chronic pain sufferers. For many patients who undergo lower back surgery, the pain only becomes more severe with increasing levels of disability. As practitioners are gaining more insight into the nature of chronic pain, they are searching for alternative treatment options that can provide safe and effective improvements in pain and function.

With the advancements made in virtual reality, augmented reality, robotics and computer graphics, we are now seeing many interactive systems which allow individuals to perform physical routines and play games, often from the comforts of their own home. The major difference between the traditional rehabilitation programs and newer simulator-based systems is that traditional machines rely heavily on the application of loads, exertion to the point of fatigue and the limits of one's strength, to be able to achieve the intended health benefits. However, virtual systems are designed to minimize strain on the user and to reduce fatigue, thereby increasing user output and accuracy to allow for a more targeted and efficient rehabilitation regime. Furthermore, traditional programs provide mainly static environments which can lead to poor engagement and compliance which is in stark contrast to virtual systems that immerse users in dynamic environments.

Examples of medical systems that utilize VR include US Patent Application No. US20150306340A1 to Giap et al., which describes a system and method monitoring a patient by positioning the patient for a predetermined medical mission; sensing biometric and physical conditions of a patient during the mission and displaying a multimedia interaction with the patient to keep the patient in a predetermined position to improve efficacy of a medical mission. These missions are meant to keep a patient engaged or immersed in the VR experience during for example radiation therapy, brachytherapy, Magnetic Resonance Imaging (MRI), angiography, biopsy procedures and endoscopy such as bronchoscopy or colonoscopy.

VR has also been used in the medical context for psychological and psychiatric needs. For example, U.S. Pat. No. 6,425,764 to Lamson describes a system and method for treating psychological, psychiatric, or medical condition by choosing a psychological strategy for treating the condition, encoding electronic instructions for a virtual reality environment in such a way that the interactive virtual reality environment implements the psychological strategy, loading electronic instructions into a virtual reality technology unit (equipped with a display for displaying the virtual reality environment and with a patient input device for receiving responses to the environment from the patient, and instructing the human patient how and when to use the virtual reality technology unit to interact with the environment).

VR has also been used in rehabilitative therapy. For example, patent application No. WO2014124002A1 to Rebstock et al., describes a system for diagnostic and therapeutic applications of gaming and media technology in which an interactive play defined by virtual augmented patient environments created in the initial patient interaction with the device. By combining rehabilitative therapy, multisensory therapy, and multidisciplinary approaches with augmented reality, while simultaneously acquiring patient data for evaluation and real time therapy modulation, the patient can be examined, evaluated, and provided multisensory therapy in one place by one clinician. This can be further extrapolated outside of the clinic with a portable embodiment that provides for continued rehabilitation and neuroplasticity training, for example, in the patient's home.

In addition to the technological advancements and the use of VR for treating various ailments, clinical research has led many to realize that the human brain is where the pain is amplified and by targeting the brain's neural pathways, the current system treats chronic pain rather than masking it with pain medications. Further advances in telehealth and telemedicine have also opened up the landscape such that patients can perform routines and even be diagnosed from their own home.

A need exists to for a system that can rehabilitate patients with pain and dysfunction using gaming technology, increase patient engagement, and neurocognitive science.

### SUMMARY OF THE INVENTION

The following summary of the invention is provided in order to provide a basic understanding of some aspects and features of the invention. This summary is not an extensive overview of the invention and as such it is not intended to particularly identify key or critical elements of the invention or to delineate the scope of the invention. Its sole purpose is to present some concepts of the invention in a simplified form as a prelude to the more detailed description that is presented below.

To achieve the foregoing and other aspects and in accordance with the purpose of the invention, a system and method for optimizing pain and physical rehabilitation through the use of virtual reality or augmented reality systems is presented. The system, method, and computer program product presented herein merges physiological, neurological, movement, and functional data to provide active biofeedback using a front-line, minimally invasive system, that can be customized to each individual patient's treatment requirements to maximize rehabilitation without overly stressing the patient.

The present invention describes a system and method that utilizes new and useful approaches in virtual reality (VR) augmented reality (AR), gamification, machine learning, and telehealth or telemedicine to maximize results for patients. The systems and method described herein is predicated upon, in part, retraining the brain by engaging emotional and behavioral neural circuits at the same time as motor-control circuits in parallel rather than in series; this engagement, combined with optimal distortion of reality, reengages the affective drivers of chronic pain, which can allow for pain remapping and ultimately rehabilitation. This closed loop system utilizes biofeedback and clinician monitoring together with machine learning for optimized results.

Generally, the system creates an immersive environment for the patient allowing for adjustment of perceived motion merged with actual motion in an engaging manner. The system gathers quantitative motion metrics for assessment and derive feedback for optimal rehabilitation. The system optimizes a patient's rehabilitation profile in the virtual environment utilizing machine learning and draws upon these to enable population level management of chronic pain and injuries.

In one embodiment, the system and method treats chronic pain and dysfunction using the VR system to engage, motivate, and guide patients through physical, emotional and cognitive-behavioral treatments. All three of these factors lead to chronic pain cycles and the system and games associated therewith address all three components in a multimodal, parallel fashion. The game(s) works by changing the patient's perception of movement (either by distracting or by altering visual cues “tricking the brain”) in order to “nudge” the patient to complete rehabilitation routines that normally would be perceived to be boring, painful or both. In this way, the system's configured to retrain the brain's neural circuits which serves to attenuate pain and improve functional movements. The patient is rewarded with game points as well as reports showing the patient's progress which provides positive reinforcement to further condition the body and brain to make behavioral changes and restore normal movement. This positive reinforcement loop replaces the maladaptive pain behaviors with more appropriate neural circuits (neuroplasticity). The cognitive behavioral and emotional targets/features of the game are reinforced with priming module which are done prior to, during, or after game play.

The cognitive behavioral module will educate the patient about the pathophysiology of chronic pain and outline the tenets of the cognitive behavioral approach to treating pain. It will incorporate psychological methodologies based in this approach, and may be incorporated at any part of the routine, before the routine or after the routine.

The system has at least a physical movement sensor configured to capture physiological quantitative movement data, a cognitive behavior sensor configured to capture brain activity data. The physiological data sensor to capture stress response, and brain activity data and combines data and sends it the routine modification module.

The emotional targeted modules focuses on teaching mindfulness breathing and meditative techniques to decrease anxiety and stress. Biofeedback from the physiologic monitors will allow patients to see how successful they are with these techniques.

The system utilizes HIPPA compliant telemedicine techniques to allow the patient to rehabilitate remotely, but under the supervision of a care provider, while utilizing real time video feedback that allows the care provider to change routines.

The system further comprises a prescription module which allows the medical professional to input prescriptions from a remote location, while using the quantitative motion and physiologic metrics feedback provided by the system to update the prescription in real time.

In this way, an objective of the present invention is to provide a new and improved system and method for treating and rehabbing chronic pain and/or restoring function from injuries.

Another objective of the present invention is to provide a new and improved system and method for making real-time adjustments to the system to ensure the rehabilitation is achieving the greatest result for the specific user.

Another objective of the present invention is to utilize the technological advances made in data mining and machine learning technologies to increase the efficiency and effectiveness of the system beyond what is currently possible.

Another object of the present invention is to provide a new and improved system and method that is easy and inexpensive to construct.

Accordingly, it is an object of the present invention to provide a new and improved system and method that serves multiple purposes and is convenient and easy to use.

In exemplary embodiments, a system and method utilizing virtual reality environments having a user use certain prescribed rehabilitation routines to treat chronic pain and/or restore function is provided. The system collects biometric and physiological data about the user relating to their performance of the specific routines or games in the virtual reality (or augmented reality) environment and as progress is made, allows for real-time adjustments to the routine programs to optimize the patient's rehabilitation process through the output of various sensors and data, whilst tracking the individual's performance data to determine how beneficial the routine was on a first endeavor or undertaking of that routine (i.e. a “first run”). The system then utilizes the output from the biometric and physiological data to a scale of success to optimize a second endeavor or undertaking (i.e. a “second run”), and so on—each time optimizing the next run using machine learning techniques. In this way, the environment can use machine learning technology to train the system using past performance data for routines deemed successful based on an objective score to create more effective rehabilitation routines plans. This data can then be pooled for different types of pain, injuries or body types (e.g., low back, shoulder, knee, women, men, exceeding certain weight, certain height, etc.) to optimize rehabilitation programs for different types of injuries based on the body type, gender, and other factors.

In exemplary embodiments, a network-based rehabilitation system for treating pain or physical dysfunction of a user utilizing virtual reality environment is disclosed. The system comprises one or more virtual reality device in communication with a network, at least one sensor in communication with the virtual reality device and the network, and a virtual reality server is in communication with the virtual reality device. The virtual reality device is configured to enable a user to interact with a virtual reality environment to execute a gamified rehabilitation program. Then at least one sensor is configured to capture movement of the user, a condition of the user, or both. The virtual reality server comprises a comprises a processor and a memory unit. The memory unit comprises a set of program modules executed by the processor. The set of program modules comprises the prescription input module, the virtual reality module, the progress analysis module, the routine modification module, a group routine module, a feedback module. The prescription input module is configured to receive an input, wherein the input comprises the gamified rehabilitation program.

The virtual reality module is configured to build the virtual reality environment respective of the input to execute the gamified rehabilitation program. The progress analysis module is configured to analyze a rehabilitation program performance of the user. The routine modification module is configured to adjust the gamified rehabilitation program based on the performance of the user. The group routine module is configured to enable at least two users to interact with the virtual reality environment to execute the gamified rehabilitation program. The feedback module is configured to provide feedback on the performance of the user. In one embodiment, the user is at least one of a patient or a healthcare provider. In one embodiment, the at least two users are at least two patients. In one embodiment, the at least two users are the patient and the healthcare provider.

In exemplary embodiments, the present invention provides a network-based rehabilitation system for treating ailments of a user utilizing an enhanced reality environment. The system comprises an enhanced reality device wearable by the user, the device being in communication with a network and configured to enable a user to interact with the enhanced reality environment to execute a rehabilitation routine; at least one sensor configured to capture biometrics of the user, a condition of the user, or both, wherein the at least one sensor is in communication with the enhanced reality device and the network; a progress analysis module configured to analyze a rehabilitation routine performance of the user, and a routine modification module configured to adjust the rehabilitation routine based on the performance of the user, wherein the routine modification module executes a machine learning algorithm and recommends a routine adjustment based on the machine.

In exemplary embodiments A network-based rehabilitation method for treating ailments of a user utilizing an enhanced reality environment is provided. The method comprises the steps of receiving, at a virtual reality server via a prescription input module, an input, wherein the input comprises a rehabilitation routine; outputting, at the virtual reality server, the virtual reality environment respective of the input to execute the gamified rehabilitation routine; analyzing, at the virtual reality server via a progress analysis module, a rehabilitation routine performance of the user, and adjusting, at the virtual reality server via a routine modification module, the rehabilitation program based on the performance of the user, wherein the routine modification module executes a machine learning algorithm and recommends a routine adjustment based on the machine.

While the present invention is described with relation to rehabilitation and treating pain, a skilled artisan will realize that the present invention can be used to track user movements, games for leisure, and the like.

Other features, advantages, and aspects of the present invention will become more apparent and be more readily understood from the following detailed description, which should be read in conjunction with the accompanying drawings.

### BRIEF DESCRIPTION OF THE DRAWINGS

FIG. 1 is a schematic drawing of an embodiment of a virtual reality rehabilitation system, including a personal computer being on-line communicating to a server, virtual reality headsets, motion analysis cameras, virtual reality controller, physiology sensor and a headphone, in accordance with one embodiment of the present invention;

FIG. 2 is a flow diagram illustrating an operation of the virtual reality rehabilitation system shown in FIG. 1, in accordance with one embodiment of the present invention;

FIG. 3 is a diagram of an embodiment showing a computer system associated with a virtual reality rehabilitation system shown in FIG. 1, in accordance with one embodiment of the present invention.

FIG. 4 is a perspective view of an optional embodiment in which a user may utilizing the virtual reality rehabilitation system shown in FIG. 1.

FIG. 5 is a perspective view of an optional embodiment in which two users may utilize the virtual reality rehabilitation system shown in FIG. 1.

FIG. 6 is a schematic drawing of an embodiment of a virtual reality rehabilitation system, including a virtual reality device associated with a patient, a virtual reality server, a communication network and a VR server according to one embodiment of the present invention.

FIG. 7 is a schematic drawing of an embodiment of a virtual reality rehabilitation system, including at least one virtual reality device associated with a patient, a user device associated with a healthcare provider, a virtual reality server, a communication network and a VR server according to one embodiment of the present invention.

FIG. 8 is a schematic drawing of an embodiment of a virtual reality rehabilitation system, including at least one virtual reality device associated with a first user, at least one virtual reality server associated with a second user, a user device associated with a healthcare provider, a virtual reality server, a communication network and a VR server according to one embodiment of the present invention.

FIG. 9 is a block diagram of a virtual reality server, according to an embodiment of the present invention.

FIG. 10 is a flowchart illustrating a network-based rehabilitation method for treating pain of a user utilizing virtual reality environment, according to one embodiment of the present invention.

FIG. 11 is an example of a random forest approach to automatically modify the routine to increase positive results for the user, according to one embodiment of the present invention.

FIG. 12 is a block diagram showing a multimodal approach for treating ailments, according to one embodiment of the present invention.

FIG. 13 is a block diagram showing a closed loop system for treating ailments, according to one embodiment of the present invention.

FIG. 14 is a flowchart illustrating a network-based rehabilitation method for treating pain of a user utilizing virtual reality environment, according to another embodiment of the present invention (shown as FIG. 14A and FIG. 14B).

FIG. 15 is a graphical user interface for providing prescription by the physician to the patient, according to one embodiment of the present invention.

### DETAILED DESCRIPTION OF THE PREFERRED EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described are shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be also understood to refer to functional equivalents of such structures. The present invention will now be described in detail with reference to embodiments thereof as illustrated in the accompanying drawings.

As used herein, the term “healthcare provider”, “medical professional” or “medical practitioner” or any persons that is aiding the user in their health means, is shall mean any person having training in the particular field the system is being used. It may mean a physical therapist, medical doctor, pain management professional, and the like.

As used herein, “pain” or “chronic pain” may also refer to physical restoration. As an example, if a system or game is treating pain, it is concurrently restoring physical movement.

As used herein, the term “prescription” shall mean an instruction written by a medical practitioner that authorizes a user to be provided a treatment. For purposes of this system, all prescriptions shall be in a digital format that can be inputted to, and read by, electronic devices.

As used herein, the term “user” shall mean any individual who utilizes the system and methods described herein for rehabilitative routine purposes.

As used herein, the term “VR” shall mean virtual reality or augmented reality.

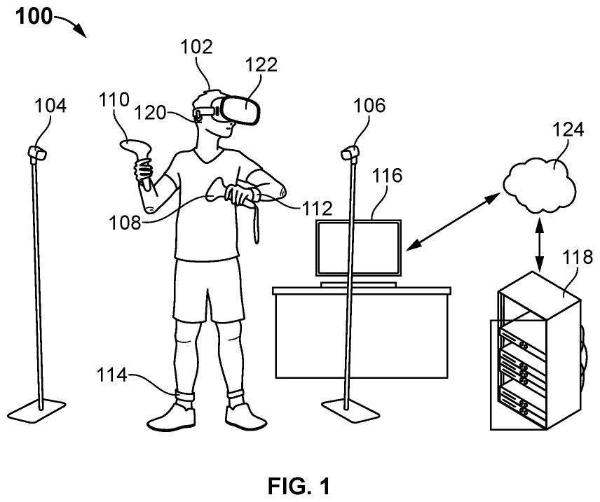

Referring now to FIG. 1, a perspective view of an individual using the virtual reality rehabilitation system 100. The system may comprise a personal computer having graphical user interface (GUI), the computer or processor being connected to a network, a server, virtual reality headsets, motion analysis cameras, virtual reality controller, physiology sensor and a headphone in accordance with one embodiment of the present invention. FIG. 1 illustrates one embodiment of the invention utilizing a complete virtual reality environment. The system 100 as shown merges physiologic and functional data to provide active biofeedback that can be customized to each individual or user's patient's treatment requirements to maximize rehabilitation without overly stressing the patient.

In this embodiment, the user 102 sees only the computer-displayed image of a virtual environment. In optional embodiments, however, a different environment may be used—namely, one that is not completely virtual such that the user 102 may see both the computer-displayed images as well as certain images from the real environment (e.g., augmented reality) in which user's experience digital play in a real-world environment. In optional embodiments, machines typically associated with the type used to perform rehabilitation (e.g., stationary bicycles, treadmills, rowing machines) may be incorporated such that the user 102 is able to see the user's arms and hands as well as portions of the physical machine, as well as the virtual environment. For example, an individual conducting rehab using a bicycle may see the physical bicycle they may be sitting on as well as their arms operating the bicycle, with a computer-displayed image such as a road that the user will ride the bicycle on.

Referring now to FIG. 1, the user 102 may incorporate one or multiple VR controllers 108 and 110 in the routine that is prescribed. The VR controllers 108 and 110 allow the user to interact with the virtual environment. For example, the VR controllers 108 and 110 may allow the user to simulate swinging a baseball bat, or holding the handles of a bicycle or holding a golf club. An example of a VR device usable with the present system is the HTC VIVE® In the current embodiment, the user 102 is holding two VR controllers 108 and 110. In optional embodiments, the user 102 may only hold one of the two possible VR controllers. The user 102 may also wear a VR headset 122 having a partially transparent projection screen that covers the eyes of the user 102. A headphone 120 may also be coupled to the VR headset 122 to provide for accompanying audio. For example, if the user 102 is swimming in the virtual reality rehabilitation routine, then the headphone 120 provides the sounds typically heard while swimming such as the splashing of water in order to provide a more immerse virtual routine experience and to create a positive environment. In optional embodiments, the headphone 120 may also be coupled to a microphone that will allow the user 102 to use voice commands or to communicate with other participants in the virtual reality rehabilitation routine. For example, if the user is running in its virtual routine, then the user 102 may be able to use a voice command via the microphone to communicate with the system to request information such as duration of time spent running or calories burned; in optional embodiments, the user 102 may make requests to alter the routine such as stop or slow down. In optional embodiments, the user 102 may communicate with other participants if participating in a group routine such as playing a group sport or conducting group yoga.

Still referring to FIG. 1, motion and biometric analysis cameras 104 and 106 are placed proximate to the user 102 and continuously gather biometrics information as the user 102 is performing the routine or prescription. The processor 116 is configured to convert the biometric information into an output indicative of the statistical analysis of the user's physical and behavioral movements. In other words, it is configured to conversion motion into usable data. Each user or patient's biometric signatures and analysis may be entered into a database (anonymously if required) which allows pooling of information to be electronically accessed.

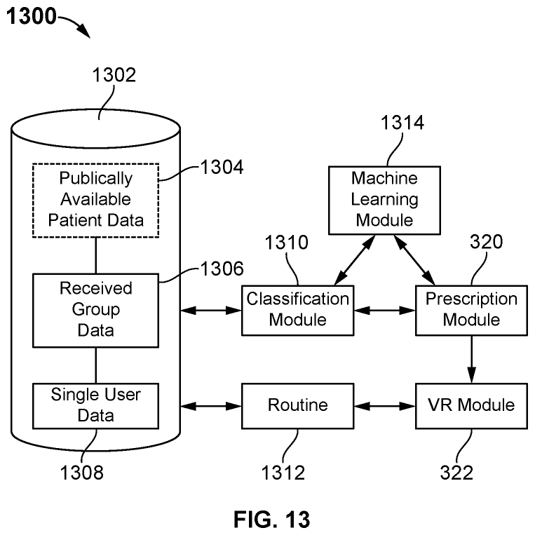

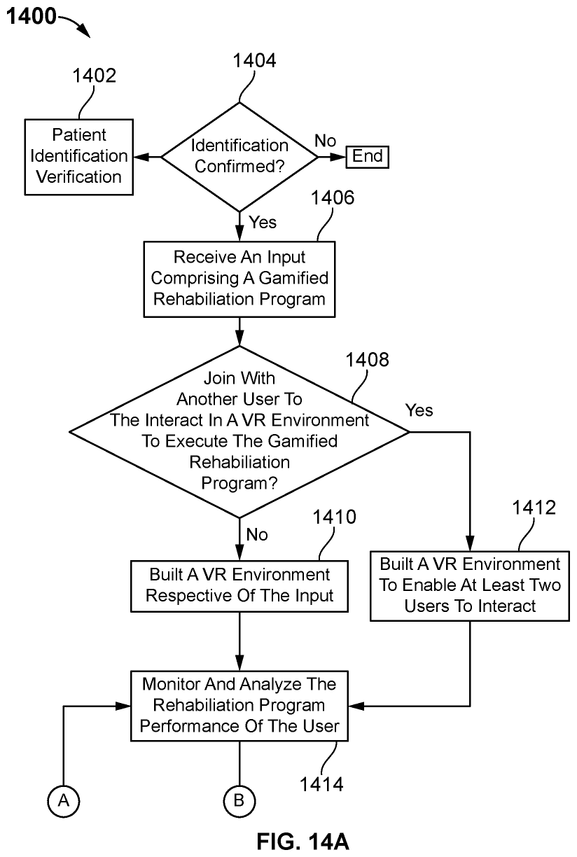

Still with reference to FIG. 1, various physiological sensors 112 and 114 are coupled to the user and allow for physiological data measurements. In the current embodiment, there are only two physiological sensors 112 and 114 depicted, but in optional embodiments more than two sensors may be coupled to the system. The sensors may measure Muscle Tension (MC), Heart Rate (HR), Finger Temperature (FT), Respiration Rate (RR), Carbon dioxide (CO2) and Oxygen Saturation (SpO2). A sensor vest may also be used to measure the movement in the trunk or extremity of a user. As an example, pulse oximetry, which provides a noninvasive procedure for measuring the oxygen status of circulating blood may be used. The system may also utilize brain sensing technology to measure neurotransmission. The data gathered by these various physiologic sensors are processed at a processor 116, and sent over a network 124 to be housed at a server 118. These data sets are used to forward a patient's rehabilitation, discussed later with reference to FIG. 13.

With reference still to FIG. 1, the motion analysis devices 104 and 106 are coupled to a processor 116, such that all of the data gathered by the cameras or biometric devices are transferred to the processor 116 where it can be stored, monitored and analyzed in real time to allow for real time modifications to the system to better the experience. In the current embodiment, the motion analysis cameras 104 and 106 are shown coupled to the processor 116 through the use of WIFI, Bluetooth® or other wireless means. In optional embodiments, however, the motion analysis cameras 104 and 106 may be coupled to the processor 116 via a wireless interface. In the current embodiment, the VR device is a personal computer, but in optional embodiments, the processor 116 may be incorporated into a laptop, smart phone tablet, ne, interactive kiosk or any similar type of device with processing power necessary to operate the system. In addition, the processor 116 accepts inputs from both the user and the medical professional to input the suggested prescription, select specific rehabilitation routines that range from rehabilitation based on physical injury, to pain management generally. The system provides the ability real-time modifications to the prescription either using machine learning or manual user input. The processor 116 also allows a prescribing physician to directly input the prescription, select specific workouts, monitor the user's 102 performance in real time, and make adjustments to the prescription and routine accordingly. In optional embodiments, the VR device 116 also allows the user to connect to a network and other users who they can in turn utilize an interactive group session (e.g., a playing a team sport such as basketball, baseball or football, go on a run together) and work with. The processor 116 is coupled to a VR server 118 via a network interface which allows the prescribing physician to also monitor and analyze the user's 102 data and make changes to the prescription. In optional embodiments, the prescribing physician will also be able to perform remote and real-time assessments via the processor 116. In even other optional embodiments, the prescribing physician can also make in-person real time assessments if the system is located in the physician's office. Generally, the various sensors and cameras illustrated in FIG. 1 may be connected to the VR device via cables, some of which are not included in the drawing.

In this way, the processor 116 may be connected to other server arrays and cloud systems such that the system is connected to administration, billing or other systems, via a communication link network 124 of data processing devices such as a LAN, WAN, the Internet, or combinations thereof. In particular, the communication link may be to e-mail systems, fax, telephone, wireless communications systems such as pagers and cell phones, wireless PDA's and other communication systems. The communications systems may also be HIPPA compliant so that patients and medical professionals may communicate over the network (e.g., telehealth). As such, the communication link is capable to execute various communications protocols in order to establish and maintain HIPPA compliant communication with the processor 116.

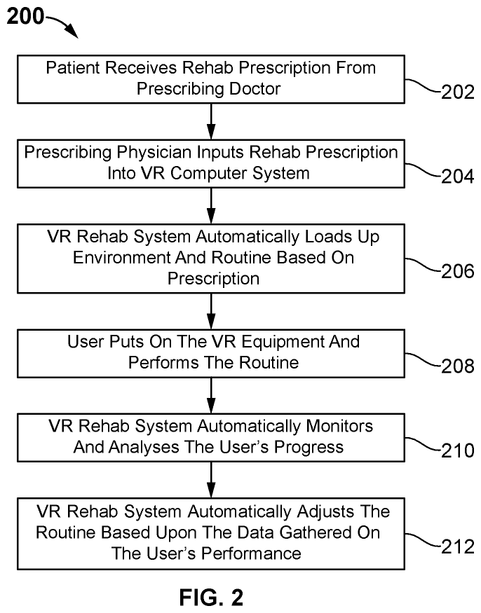

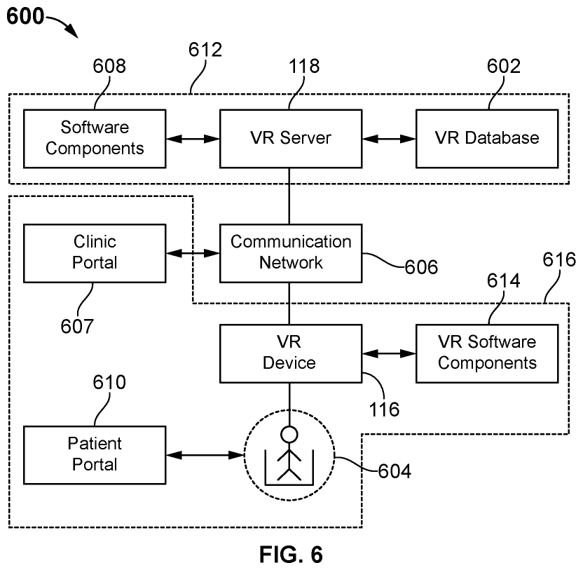

Referring now to FIG. 2, a flow diagram illustrating a process for the virtual reality rehabilitation system to provide a user with a rehabilitation routine, monitor such routines and make real-time adjustments using either machine learning or manual input, is presented generally at 200. As shown, the process begins with a user visiting a prescribing physician who evaluates the user and issues a physical therapy prescription to the user step 202 based on the evaluation. In optional embodiments, the user may not have to obtain a prescription or evaluation from a physician but may be able to configure their own rehabilitation routines based upon prior data gathered in the system. Once the user has been evaluated and a prescription has been issued, the prescribing physician will then input the prescription directly into the VR computer system step 204. In optional embodiments, the user will be able to input the prescription itself without needing the assistance of the prescribing physician. After the prescription is input, the virtual reality rehabilitation system will automatically compile the routine and build the virtual environment based on the prescription step 206. In optional embodiments, the user may be able to select the routines based upon certain target goals determined by the prescribing physician. Thereafter, the user will equip the VR headset 122, headphone 120, VR controllers 108 and 110, and physiology sensors 112 and 114, all of which were described in detail in relation to FIG. 1 and begin performing the prescribed routines step 208. While the user is performing the routines, the virtual reality rehabilitation system will automatically monitor and analyze the user's progress step 210 via the motion analysis camera 104 and 106 and the sensors 112 and 114. Once the routine data is analyzed, the system will make automatic adjustments to the routine based upon the data gathered regarding the user's performance step 212. For example, if the user received a prescription that required the user to walk a mile in fifteen minutes, and the system gathers the data and determines that because the user will be able to walk the mile in less than fifteen minutes, then the user should walk two miles in twenty minutes. In another example, if the user received a prescription to perform specific routines to strengthen certain muscles as part of a rehabilitation program, and the system determines that those muscles have reached the peak strength for what the user can output at the current time, then the system may adjust the prescription and assign a separate task to work out different muscles to prevent against over exertion and foster efficient rehabilitation. In optional embodiments, the prescribing physician may make adjustments in real time to the routine based upon the user's performance. In further optional embodiments, the prescribing physician may make adjustments to the routine after reviewing the user's performance remotely, but not in real time. Once the user has completed the virtual reality rehabilitation routines, then the system provides feedback to the user on its performance, stores it to the VR server, and automatically communicates such results to the user's prescribing physician. In optional embodiments, there may be no need to communicate the results to a physician. The ability to control routines in a telehealth setting is discussed with relation to FIG. 6.

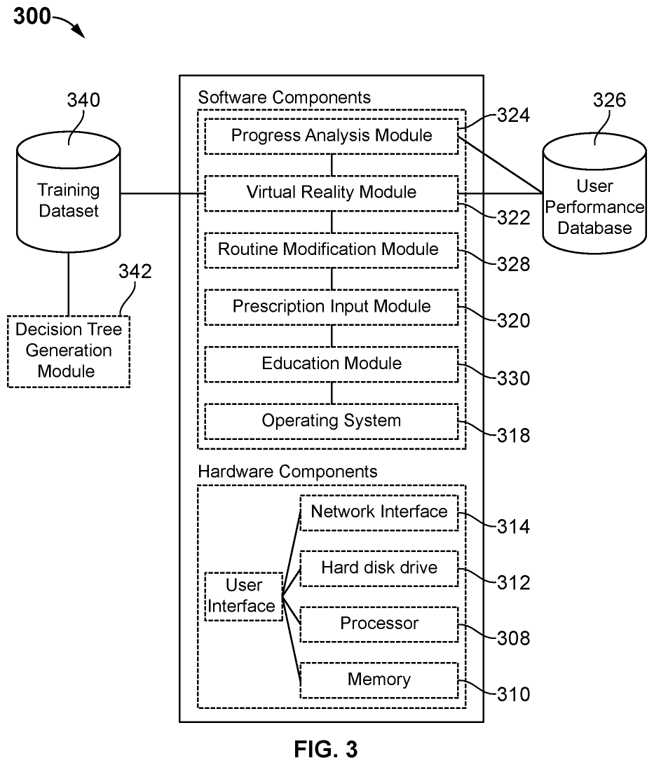

Referring now to FIG. 3, a diagram of an embodiment of a computer system that may performance virtual reality-based rehabilitation routines, is shown generally at reference numeral 300. The embodiment 300 is a simplified example of a computer environment in which a virtual reality system may operate with various modules and consuming applications. The embodiment 300 illustrates the functional components of a system. In some embodiments, the functional component may be a hardware component, a software component, or a combination of hardware and software. Some of the components may be application level software, while other components may be operating system level components. In some cases, the connection of one component to another may be a close connection where two or more components are operating on a single hardware platform. In other cases, the connection may be made over network connections spanning long distances. Each embodiment may use different hardware, software, and interconnection architectures to achieve the described functions.

Still referring to FIG. 3, the VR device 302 may have a set of hardware components and software components. The hardware components may represent an architecture of a computing device, such as a desktop or server computer. In some embodiments, the VR device 302 may be a personal computer, network appliance, interactive kiosk or other device. The VR device 302 may also be a portable device, such as a laptop computer, netbook computer, personal digital assistant, mobile telephone, or other mobile device.

Still referring to FIG. 3, in the present embodiment the hardware components may also comprise a central processing unit 308, random access memory 310, nonvolatile storage 312 and a network interface 314. The random-access memory 310 may store executable code as well as data that may be immediately accessible to the processor 308, while nonvolatile storage 312 may store executable code and data in a persistent stage. The network interface 314 may include hardwired and wireless interfaces through which the device 302 may communicate with other devices. The hardware components 304 may also include one or more user interface devices which may include monitors, displays, keyboards, pointing devices, and any other type of user interface device.

The software components 306 comprise an operating system 318 on which various applications may execute as well as user performance database 326 that contains the data gathered regarding a user's performance of any virtual reality rehabilitation routines.

A prescription input module 320 allows for the entry of a medical practitioner's prescription data file. This may occur through the use of a remote GUI in the physicians' control via a wireless network connection. Once the prescription is input, the virtual reality module 322 will build the virtual environment and the applicable routine as prescribed by the medical practitioner. Further, the prescription input module may be configured to work with education module 330 to not only build a VR or AR environment (e.g., run game software) but also to build an educational and coaching construct to teach the patient about pathophysiology of chronic pain and to teach the methods of the cognitive behavioral and mindfulness approach to treating pain during the immersive VR or AR experience. Importantly, the system takes a multimodal approach to treating the cognitive-emotional state and physical state in parallel, rather than in series.

With reference still to FIG. 3, a progress analysis module 324 collects all of the data regarding the user's performance of the prescribed virtual reality rehabilitation routine, and stores the data into the user performance database 326, and then will compute the probability that the user will be successful in accomplishing its rehabilitation goals (e.g., walk again, strengthen muscles) based upon the current prescription and the users performance thereof, all of which is stored within the training dataset 340 and then processed using a machine learning algorithms or decision tree analyses such as random forest, in which a decision tree generation module 342 to determine the likelihood of success of future routines, and set routines based on these inputs. In the optional embodiments, if a decision tree is used, the decision tree generation module 342 will generate a forest of decision trees used in a Random Forest algorithm based on the training dataset 340. Furthermore, historical performance data for generating and training the decision trees (e.g., the “training dataset”) may also be stored in the training dataset 340. In optional embodiments, a data processing module may be used to turn data stored in the user performance database 326 into quantitative features that can be analyzed by a machine learning algorithm as part of the progress analysis module 324. Once the progress analysis module 324 analyzes the user's progress, the routine modification module 328 will adjust the prescription as is necessary to ensure the virtual reality rehabilitation routines allow the user to rehab in the most efficient and effective manner.

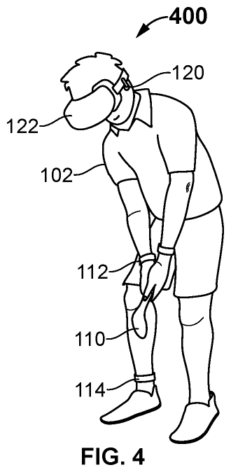

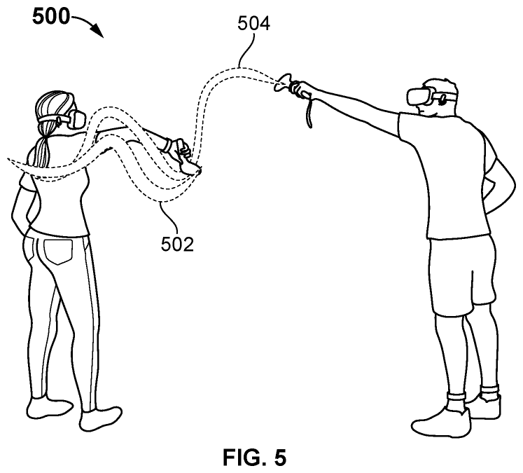

Referring now to FIG. 4, a perspective view of an optional embodiment in which a user may utilizing the virtual reality rehabilitation system shown in FIG. 1, is presented generally at 400. In this embodiment, the user 102 has equipped the headphone 120, VR headset 122, physiology sensors 112 and 114, and VR controller 110. In this embodiment, the user 102 is only using one VR controller 110. Here, we can see the user 102 performing a routine using the system. In this particular embodiment, the user 102 is performing a golf swing motion, wherein the user 102 has conformed to the traditional stance taken by a golfer as they prepare to hit a golf ball.

Referring now to FIG. 5, a perspective view of an optional embodiment in which two users may utilize the virtual reality rehabilitation system shown in FIG. 1, is presented generally at 500. In this embodiment, two separate users are connected via a wireless network and are participating in a virtual reality rehabilitation routine wherein they are playing tennis. In this embodiment, the virtual wavelength 502 and 504 are used for illustrative purposes so show how virtual environment would transport signals from one user to another user to create the interactive multi-user environment—in this particular embodiment, the wavelength is illustrative of the tennis ball that each user would be hitting in their virtual routine.

FIG. 6 is a schematic drawing of an embodiment of a virtual reality rehabilitation system 600 in which telehealth can be utilized. The schematic comprises the virtual reality device 116 associated with a patient 604, a virtual reality server 118, a communication network 606 and a VR database 602 according to one embodiment of the present invention. The system 600 is configured to enable the patient or user to feed input and provides a virtual game environment or a virtual world to perform the inputted game utilizing rehabilitation approach. The communication network 606 is a telehealth communications network includes communicatively coupled hardware and software systems that enables both HIPPA compliant real-time communication, appointment scheduling between one or more providers (e.g., physicians), customers (e.g., patients) and/or care givers. The telehealth communications network 606 is a dynamic network that enables parties to engage in a communication session on-demand for the real-time diagnosis and treatment of patients by qualified, remotely located providers. Because the communication sessions may be facilitated electronically by providing some or all participants with a customer's medical records via the telehealth communications network and is again HIPPA compliant. In embodiments, the medical practitioner has the ability to electronically prescribe routines for the customer using the telehealth communications network.

In operation, the medical practitioner may log into a clinic portal 607 to integrate various processes in connection with patient, and conferencing capabilities by communicatively coupling point-of-care sites with and care-dispensing sites and to enable on-demand healthcare. The clinic portal 607 allows the medical practitioner or other authorized access a database of health-related information or central data repository and link all the relevant medical-related information for each patient 604. Medical-related or health-related information includes but is not limited to medical history, medical profile information, lifetime laboratory reports, demographic information, current and past prescribed medications, insurance coverage information, and family medical history. In one embodiment, the patient portal 610 enables the patient to review the updates made by the medical practitioner.

In this way, the network 606 is communication with client-side infrastructure 616 and server-side infrastructure 612 which enables physicians to engage in a dynamic (e.g., on-demand) communication and collaboration with remotely located patients. The components of each the server-side 612 and client side 616 may vary, but generally comprise the elements shown therein. The network 606 provides HIPPA compliant unified communications and video conferencing capabilities (e.g., patient computers, video conference monitors, a conference telephone, video cameras such as high-resolution cameras, administrative computer and monitor, a server, a printer, a server recorder/caching device, and a router) and also through the use of various GUIs send and receive various prescriptions to the patient 604. Further, the patient can communicate information back to the physician in a HIPPA compliant manner.

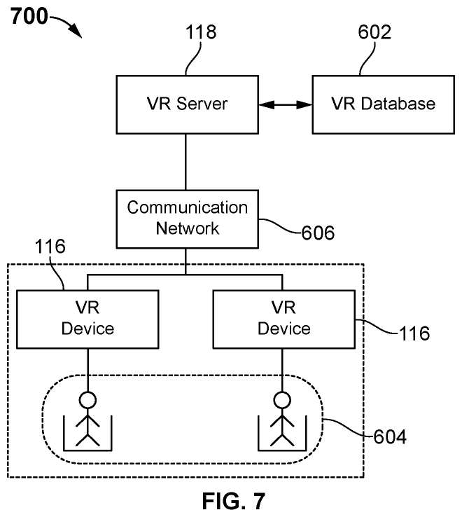

FIG. 7 is a schematic drawing of an embodiment of a virtual reality rehabilitation system 700, including at least one virtual reality device 116 associated with a first user or first patient, at least one virtual reality device 116 comprising VR software components 614 associated with a second user or a second patient, the communication network 606, the virtual reality server 118 and the VR database 602, according to one embodiment of the present invention. The system 700 is configured to couple at least two users including the first user and the second user in a single game environment 604 to perform the inputted game. In this way, multiple patients can participate in rehabilitation sessions.

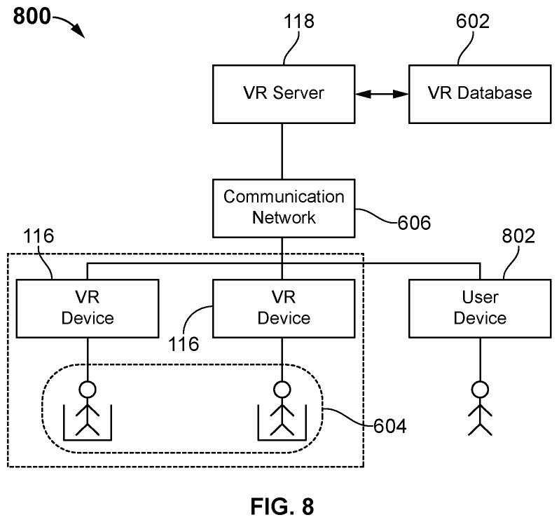

FIG. 8 is a schematic drawing of an embodiment of a virtual reality rehabilitation system 800, including at least one virtual reality device 116 associated with the patient, at least one user device 802 associated with the healthcare provider, the communication network 606, the virtual reality server 118 and the VR database 602 according to one embodiment of the present invention. The system 800 is configured to enable the healthcare provider and the patient to interact in the game environment 604. The system 800 is further configured to enable the healthcare provider to remotely monitor the patient in the game environment 604 in a HIPPA compliant manner.

In an embodiment, the user device 802 is at least one of a desktop, a laptop, a tablet, a mobile phone, and mobile and/or handheld electronic devices. In an embodiment, the user device 802 is in communication with the communication network 606 to access the VR server 604. In an embodiment, the communication network 606 could be Wi-Fi network, WiMax network, and wireless local area network. In one embodiment, the at least one VR database 602 may be accessible by the VR server 604. The VR database 602 may be integrated into the VR server 604 or separate from it. In some embodiments, the VR database 602 resides in a connected server or in a cloud computing service. Regardless of location, the database 602 comprises a memory to store and organize certain data for use by the VR server 604.

In one embodiment, the virtual reality device 116 comprises VR controllers 108 and 110, VR headset 122 coupled with a microphone, various physiology sensors 112 and 114 are coupled to the patient and the motion analysis cameras 104 and 106. In one embodiment, the VR device 116 further includes one or more sensor to track the movement of the user, which includes, but not limited to, degrees of motion, a velocity of movement, repetitions, and duration of the movement. In one embodiment, the physiology sensors 112 and 114 are configured to track data related to effort, stress response, and emotional response. More specifically, the physiologic sensors will include heart rate monitor, respiratory (breathing) rate monitor, EMG (muscle tension) sensors, EEG (brain activity) sensors, and galvanic skin (sweat) response monitors. Further, to sense motion, a sensor jacket may be worn by the user. The data from these physiologic sensors basically tells us how much effort someone is providing as well as whether or not they are under too much stress. If these parameters are elevated the system will decrease difficulty level automatically. Brain sensor may be further employed to measure response signaling, which assist in better understanding of those regions that are active during the session or routine. The movement and physiologic data are outputted in real time as well as report to the medical practitioner at the end of the session.

The world is a game environment 604 that enables the patient to perform rehabilitation routines. In one embodiment, the rehabilitation routine is a low back treatment routine. In another embodiment, the rehabilitation routine is configured to address one of a physical condition from a group including, but not limited to, knee pain, neck pain, stroke, cerebral palsy, spasticity, shoulder pain/stiffness, etc. In yet another embodiment, the rehabilitation routine includes, but not limited to, pain education, stress management, mood management (via relaxation, meditative therapies) and cognitive behavioral therapies. In yet another embodiment, the game environment utilizes a rehabilitative approach to address the physical approach. In one embodiment, the system (600, 700, and 800) is compatible with different hardware platforms.

In particular, as it relates to pain management and other pain and physical conditions such as knee pain, neck pain, stroke, cerebral palsy, spasticity, shoulder pain/stiffness, etc., each of the games that are played in the VR environment use a rehabilitative approach to treating these conditions but also incorporate other modules focused on stress, mood management (via relaxation, meditative therapies) and cognitive behavioral therapies. The brain sensors discussed in relation to FIG. 1. The systems and method described herein is predicated upon, in part, retraining the brain by engaging emotional neural circuits at the same time as motor-control circuits; this engagement, combined with optimal distortion of reality, reengages the affective drivers of chronic pain, which can allow for pain remapping and ultimately rehabilitation.

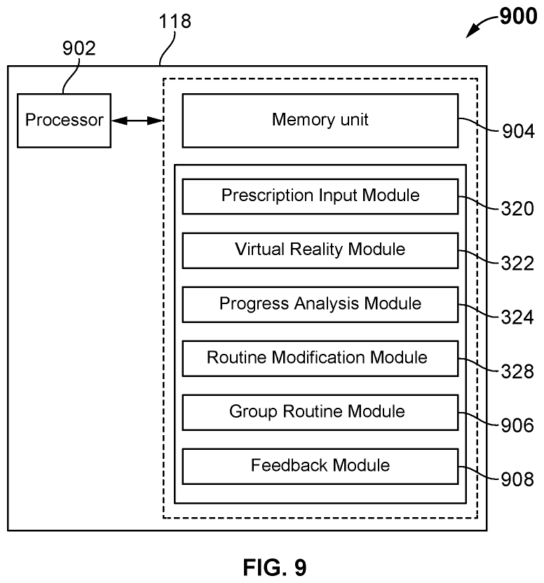

Referring to FIG. 9, a block diagram 900 of the virtual reality server 118 is provided. The virtual reality server 118 comprises a processor 902 and a memory unit 904. The memory unit 904 comprises a set of program modules executed by the processor 902. The set of program modules comprises the prescription input module 320, the virtual reality module 322, the progress analysis module 324, the routine modification module 328, a group routine module 906, a feedback module 908. The prescription input module 320 is configured to receive an input, wherein the input comprises the gamified rehabilitation program. The virtual reality module 322 is configured to build the virtual reality environment respective of the input to execute the gamified rehabilitation program. The progress analysis module 324 is configured to analyze a rehabilitation program performance of the user. The routine modification module 328 is configured to adjust the gamified rehabilitation program based on the performance of the user. The group routine module 906 is configured to enable at least two users to interact with the virtual reality environment to execute the gamified rehabilitation program. The feedback module 908 is configured to provide feedback on the performance of the user. In one embodiment, the feedback includes, but not limited to, session reports comprising movement data, physiologic data, and game success rate.

In one embodiment, the user is at least one of the patient or the healthcare provider, according to system 600. In one embodiment, the at least two users are at least two patients, according to system 700. In one embodiment, the at least two users are the patient and the healthcare provider, according to system 800. In one embodiment, the prescription input module 320 is configured to receive the input from at least one of a healthcare provider or a patient. In one embodiment, the routine modification module 328 is configured to enable at least one of the healthcare provider or patient to adjust the gamified rehabilitation program based on the performance. In one embodiment, the routine modification module 328 is configured to automatically adjust the gamified rehabilitation program based on the performance.

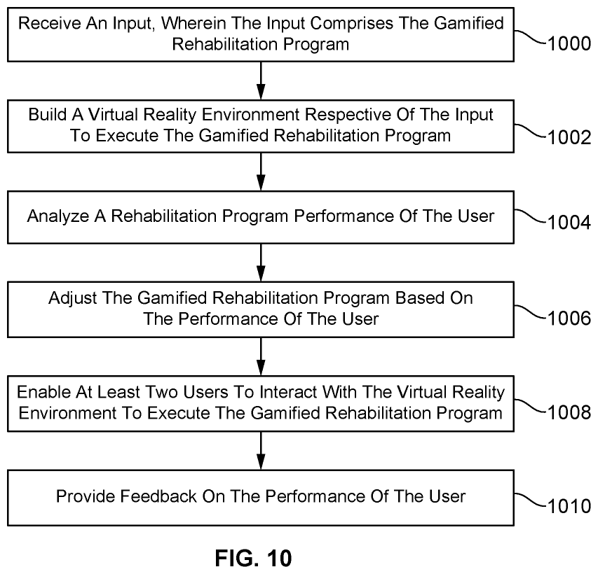

Referring to FIG. 10, in one embodiment, the present invention provides a network-based rehabilitation method 1000 for treating pain of a user utilizing virtual reality environment. The method is incorporated in a system comprising one or more virtual reality device in communication with a network configured to enable a user to interact with a virtual reality environment to execute a gamified rehabilitation program; at least one sensor configured to capture a movement of the user, a condition of the user, or both, wherein the at least one sensor is in communication with the virtual reality device and the network, and a virtual reality server in communication with the virtual reality device, at least one sensor, a communication interface via the network. The method includes the step 1000 of: receiving, at the virtual reality server via a prescription input module, an input, wherein the input comprises the gamified rehabilitation program. The method further includes the step 1002 of: building, at the virtual reality server via a virtual reality module, the virtual reality environment respective of the input to execute the gamified rehabilitation program. The method further includes the step 1004 of: analyzing, at the virtual reality server via a progress analysis module, a rehabilitation program performance of the user. The method further includes the step 1006 of: adjusting, at the virtual reality server via a routine modification module, the gamified rehabilitation program based on the performance of the user. The method further includes the step 1008 of: enabling, at the virtual reality server via a group routine module at least two users, to interact with the virtual reality environment to execute the gamified rehabilitation program. The method further includes the step 1010 of: providing, at the virtual reality server via a feedback module, feedback on the performance of the user.

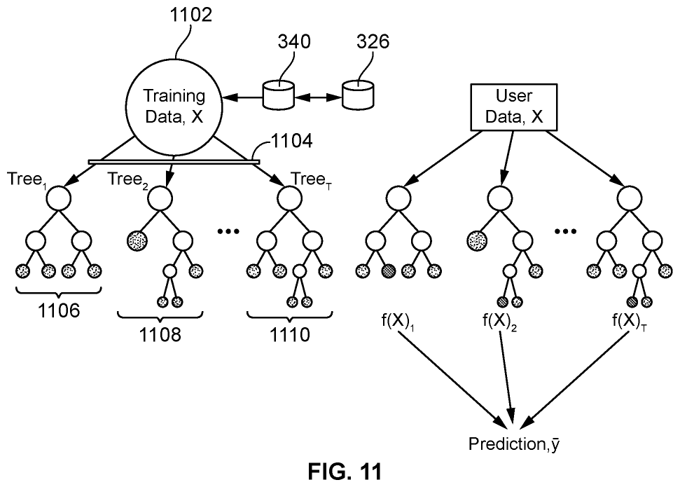

With reference now to FIG. 11, an example of a random forest approach to automatically modify the routine to increase positive results for the user. In this way, the routine modification module 328 together with the user performance data based 326 utilizes data gathered either for a single user or across predetermined categories or demographics to compute the probability that a certain routine in the database of routines will increase the medicinal effects of the routine.

In operation, to establish training data for the random forest, each routine may be given a score by the patient based on its pain reduction effect. The operator may then group certain kinds of routines based on certain scores, and other various metadata such as user age, gender, weight, and the like. Once the training data set 340 is established, it is stored within the training dataset 1102, and then processed using the decision tree generation module 1104 to determine a routine ranking depending upon the user input. The training data may come from many patients, and historical data may also be used. This is discussed in greater detail with relation to FIG. 13. In this embodiment, the trees (1106, 1108 and 1110) are trained independently by recursive binary partitioning of a bootstrapped sample of the input data, X. Once the trees are generated, the user performance data 326 (for a single user during a routine, for example, is dropped down through each tree and the response estimate is the average over the all the individual predictions in the forest).

In operation, once the routine modification module 328 analyzes the user together with the routine prioritization module will rank and present routines in order from likelihood of most impactful for the user to the least impactful for the user. It will further present a list so that the medical professional may choose between the top routines since the professional may have a deeper understanding of specifics involving the patient's symptoms.

Specifically, routine modification module 328 and tree generation module 1104 uses the RFA to build an ensemble (or “forest”) of decision trees that are used to prioritize the routines. The RFA is a non-parametric ensemble approach to machine learning that uses bagging to combine decisions of multiple classification (or decision) trees to classify data samples, in this case the routines for patients that will treat a specific ailment. More details about the RFA may be found in L. Breiman, “Random Forests,” Machine Learning 45 (1):5-32 (2001) and A. Liaw et al., “Classification and Regression by Random Forest,” R News, Vol. 2/3, p. 18 (2002), both of which are incorporated by reference. In the typical instance, the RFA either a will identify one or more datasets based on posted media and make a standard assumption on its reach based on certain data features or hey performance indicators (in this case provided by patients). The system data-mines such datasets, taking into consideration the specific patient attributes to extract a sufficient number of data within a specific category to train one or more deep learning algorithms.

The data mining needed to create a strong deep learning algorithm aims at surfacing and injecting vast amounts of data from patients automatically or semi-automatically, and therefore the decision tree generation module is configured to analyze large quantities of data to extract patterns such as groups of data records (cluster analysis), unusual data (anomaly detection), and dependencies (association rule mining, sequential pattern mining). This usually involves using database techniques such as spatial indices. These patterns can then be seen as a kind of summary of the input data, and may be used in further analysis or, for example, in machine learning and predictive analytics.

In this way routines can be ranked by scoring effectiveness based on ailment X, Y, or Z (n+1) ranging from 0.0 for little to no effectiveness to a maximum of 1.0 for maximum effectiveness, as one example.

In optional embodiments, deep learning recurrent neural networks may use hidden computational nodes and various gates and may be self-tuning or user-tuning, in some embodiments. After the process of tuning, the algorithm will be evaluated to assess the degree to which it accurately identifies the media test data it has never encountered with the “vector space” it has been trained to recognize. This, over time, improves accuracy and patient success.

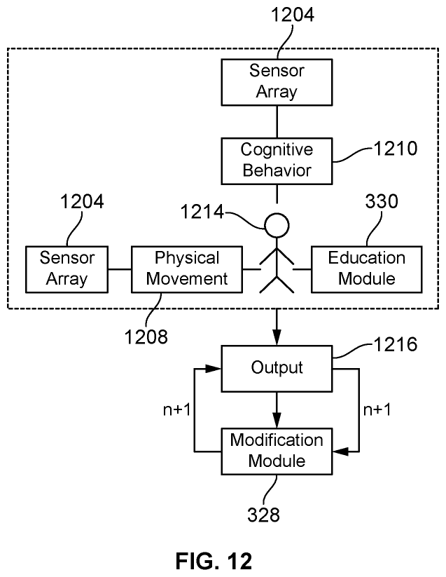

Referring now to FIG. 12, a block diagram showing a multimodal approach to treating ailments such as pain and improving muscle and joint functions shown. As shown, the system combines multiple data inputs from sensor array 1204 directed toward user 1214 cognitive behavior 1210, sensor array 1204 direct toward user 1214 physical movement 1208, and also a teaching tool that is used in the routine, via education module 330. These modules are used by the system in a multimodal manner, meaning they are run in parallel to alleviate symptoms for pain and/or increase physical range of motion. The data inputs from each module are output 1216 in the form for a report, for example, that a physician can use (in real time). The system however, utilizes machine learning to run the data through a continuous loop thereby utilizing routine modification module 328 providing information to the physician as to which prescription out of a predetermined group of prescriptions has the best chance of success with that user 1214.

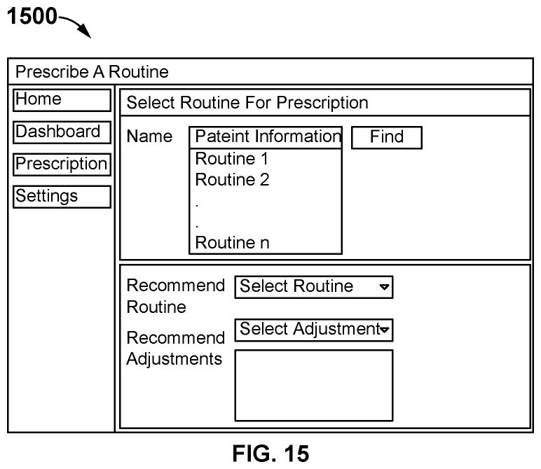

Referring now to FIG. 13, a block diagram 1300 showing a closed loop system for treating ailments such as pain and improving muscle and joint function is shown. The closed loop system includes a database 1302, a machine learning module 1324, classification module 1310, a prescription module 320, routine 1312 and a VR module 322. The database 1302 stores publicly available patient data 1304, a received group data 1306 and a single user data 1308. The prescription module 320 enables the physician to enter the prescription data of the patient, that could be implemented utilizing the VR module 322. Referring to FIG. 15, is a graphical user interface 1500 for providing prescription by the physician to the patient, according to one embodiment of the present invention. The prescription module 320 enables the physician to select the routine the list of preloaded prescriptions. In another embodiment, the prescription module 320 also recommends prescription based on the machine learning output. The prescription module 320 also enables the physician to view outputs showing the physician patient data from the newly listed sensors.

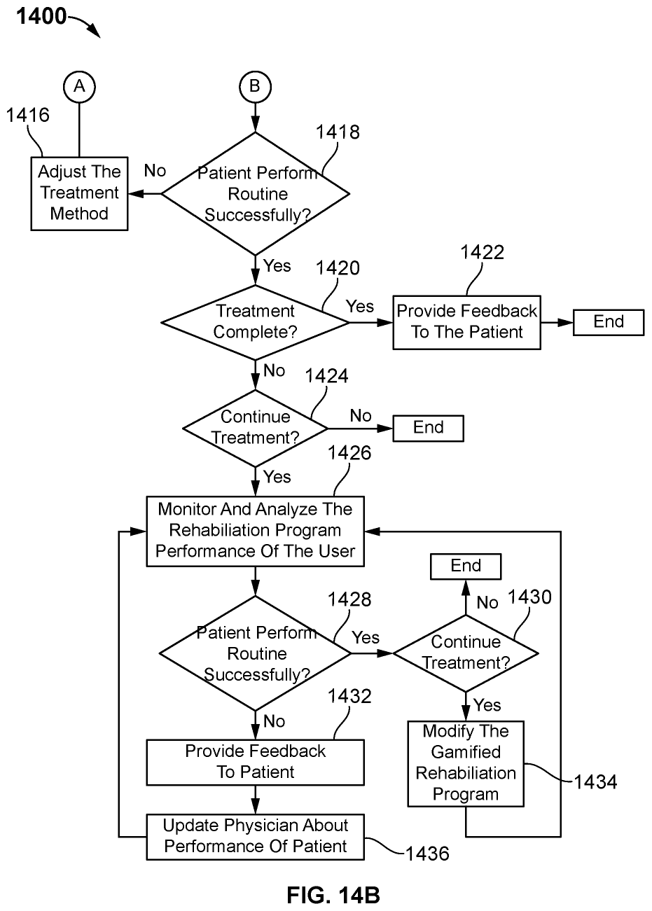

FIG. 14 is a flowchart 1400 illustrating a network-based rehabilitation method for treating pain of a user utilizing virtual reality environment, according to another embodiment of the present invention. At step 1402, the patient is enabled to input patient identity. At step 1404, identification verification is done by the system. If the patient identity is not verified, the process ends. If the patient identification is verified, the system receives an input, wherein the input comprises the gamified rehabilitation program at step 1406. At step 1408, the user or patients selects to interact with another user or patient in the virtual environment. At step 1412, a VR environment is built to enables at least two users to interact or perform the gamified rehabilitation program. At step 1410, a VR environment is built respective of the input received by at step 1406 to execute the gamified rehabilitation program. In embodiments, the VR environment is selected from pre-existing routines.

At step 1414, the rehabilitation program performance of the user is monitored and analyzed. Based on the analysis, the system checks if the patient performed the routine successfully, at step 1418. At step 1416, if the performance of the patient is not ideal, a treatment method is adjusted. At step 1420, if the routine is performed successfully, the user is queried for ending the treatment (at step 1420) or for continuation of treatment (at step 1424). At step 1422, at the end of treatment feedback is provided to the patient 1422. At step 1426, on continuation of treatment, the rehabilitation program performance of the user is monitored and analyzed. At step 1428, the system checks if the patient performed the routine successfully and queried for continuation of treatment (at step 1430). If the user shows poor performance, feedback is provided to the user and the physician is updated about the performance of the patient (at step 1436). At step 1434, on selection of continuation of treatment, the system modifies the gamified rehabilitation program to improve the performance of the user.

Specific configurations and arrangements of the invention, discussed above regarding the accompanying drawing, are for illustrative purposes only. Other configurations and arrangements that are within the purview of a skilled artisan can be made, used, or sold without departing from the spirit and scope of the invention. For example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures.

While the present invention has been described in connection with what are presently considered to be the most practical and preferred embodiments, it is to be understood that the present invention is not limited to these herein disclosed embodiments. Rather, the present invention is intended to mobile phone the various modifications and equivalent arrangements included within the spirit and scope of the appended claims.

Although specific features of various embodiments of the invention may be shown in some drawings and not in others, this is for convenience only. In accordance with the principles of the invention, the feature(s) of one drawing may be combined with any or all of the features in any of the other drawings. The words “including,” “comprising,” “having,” and “with” as used herein are to be interpreted broadly and comprehensively and are not limited to any physical interconnection. Moreover, any embodiments disclosed herein are not to be interpreted as the only possible embodiments. Rather, modifications and other embodiments are intended to be included within the scope of the appended claims.

1. A network-based rehabilitation system for treating ailments of a user utilizing an enhanced reality environment, the system comprising:
an enhanced reality device wearable by the user, the device being in communication with a network and is configured to enable a user to interact with the enhanced reality environment to execute a rehabilitation routine for treating a diagnosed condition;
at least one sensor to capture biometrics of the user, a condition of the user, or both, wherein the biometrics of the user comprise anxiety, stress related responses, or both, in response to rehabilitation routine, wherein the at least one sensor is in communication with the enhanced reality device and the network;
a progress analysis module to analyze a rehabilitation routine performance of the user, wherein the routine performances of the user comprises the anxiety, stress related responses, or both, and
a routine modification module to automatically adjust the rehabilitation routine based on the performance of the user, wherein the routine modification module executes an algorithm and recommends a routine adjustment based on the algorithm to balance user rehabilitation gains related to anxiety, stress related responses, or both.

2. The system of claim 1, further comprising an education module to teach a user a cognitive behavioral and mindfulness-based approach to managing the diagnosed condition, wherein the education module is ran at various times before, during, or after game play sessions, wherein the diagnosed condition is chronic pain.

3. The system of claim 1, wherein the at least one sensor comprises:
at least a physical movement sensor configured to capture quantitative movement data;
at least a cognitive sensor configured to capture brain activity data;
at least a physiological data sensor to capture stress response;
and the combined data is received by the routine modification module;
wherein the routine modification module measures the user's stress response, the brain activity, the movement data, or any combination thereof, in response to rehabilitation routine; and
wherein the

[symbol]

4. The system of claim 1, wherein the routines are gamified routines and together with the sensors and routine modification module, are configured to engage, motivate, and guide the user through physical, emotional and cognitive rehabilitation to retrain of the brain's neural circuits which serves to attenuate the pain and improve functional movements.

5. The system of claim 1 further comprising a virtual reality server in communication with the virtual reality device, at least one sensor, a communication interface via the network, wherein the server comprises:
a virtual reality module configured to build the virtual reality environment respective of the input to execute the gamified rehabilitation program;
a prescription input module configured to receive an input, wherein the input comprises the gamified rehabilitation program.

6. The system of claim 5, wherein the prescription input module comprises a plurality of routines, and is configured to receive inputs from an acting physician, a healthcare provider, the routine modification module, or any combination thereof, to thereby upload the preloaded routine, and is further configured to rank the routines based on what the routine modification module output for the best chance of success at rehabilitation.

7. The system of claim 1, further comprising:
a database comprising user data and group data; and
a machine learning module that utilizes the user data and group data to suggest a routine for a user;
wherein the stored user data is for multiple routines for a single user, and the machine learning module optimizes a future routine.

8. The system of claim 1, wherein the server further comprises a group routine module to enable at least two users to interact with the virtual reality environment to execute the gamified rehabilitation program.

9. The system of claim 1, wherein the prescription input module receives the input from at least one of healthcare provider or user.

10. The system of claim 1, wherein the routine modification module is enables at least one of the healthcare providers or the patient to adjust the gamified rehabilitation program based on the performance.

11. The network-based rehabilitation system of claim 1, wherein the routine modification module automatically adjusts the gamified rehabilitation program based on the performance.

12. The system of claim 1, wherein the virtual reality device comprises one or more VR controller or headphones, a VR headset coupled with microphone, one or more physiology or motion sensors are coupled to the patient and one or more motion analysis cameras coupled to the VR device.

13. The network-based rehabilitation system of claim 1, further comprises one or more database to store a publicly available patient data, a received group data and a single user data.

14. A network-based rehabilitation method for treating ailments of a user utilizing an enhanced reality environment, the method comprising the steps of:
receiving, at a virtual reality server via a prescription input module, an input, wherein the input comprises a rehabilitation routine;
outputting, at the virtual reality server, the virtual reality environment respective of the input to execute the gamified rehabilitation routine for treating a diagnosed condition;
analyzing, at the virtual reality server via a progress analysis module, a rehabilitation routine performance of the user using captured biometrics of the user, a condition of the user, or both, wherein the biometrics of the user comprise anxiety, stress related responses, or both, in response to rehabilitation routine; and
automatically adjusting, at the virtual reality server via a routine modification module, the rehabilitation program based on the performance of the user, using the anxiety, stress related responses, or both of the user, wherein the routine modification module executes an algorithm and recommends a routine adjustment based on the response to balance user rehabilitation gains related to anxiety, stress related responses, or both.

15. The method of claim 14, further comprising educating the user about cognitive behavioral and mindfulness based approach to managing the diagnosed condition via an education module is configured to run at various times before, during, or after game play sessions, wherein the diagnosed condition is chronic pain.

16. The method of claim 14, wherein the routines are gamified routines and together with a plurality of sensors and routine modification module, are configured to engage, motivate, and guide the user through physical, emotional and cognitive rehabilitation to retrain user's neural circuits which serves to attenuate the pain and improve functional movements.

17. The method of claim 14, wherein the user is at least one of a patient or healthcare provider.

18. The method of claim 14, wherein the virtual reality device comprises one or more VR controllers, a VR headset coupled with a microphone or headphones, one or more physiology or motion sensors are coupled to the patient and one or more motion analysis cameras coupled to the VR device.

19. The method of claim 14, further comprising a step of:
receiving, at the virtual reality server via the prescription input module, the input from at least one of the healthcare provider or patients;
executing a machine learning module that utilizes the user data and group data to suggest a routine for a user;
wherein the stored user data is for multiple routines for a single user, and the machine learning module optimizes a future routine.

20. The network-based rehabilitation method of claim 14, further comprising a step of: enabling, at the virtual reality server via the routine modification module, at least one of the healthcare provider or patients to adjust the gamified rehabilitation program based the user's stress response, the brain activity, the movement data, or any combination ther [symbol]
